(19) United States
(12) Patent Application Publication
(10) Pub. No.: US 2025/0019648 A1
(43) Pub. Date: Jan. 16, 2025

(54) A SPERM EXTENDER MEDIUM

(71) Applicant: MEAT & LIVESTOCK AUSTRALIA LIMITED, NORTH SYDNEY, SYDNEY, NEW SOUTH WALES (AU)

(72) Inventors: ZAMIRA GIBB, NEW LAMBTON (AU); ERIN KLEIN, YORK, NE (US); ROBERT JOHN AITKEN, COOKS HILL (AU)

(21) Appl. No.: 18/700,543

(22) PCT Filed: Oct. 13, 2022
(86) PCT No.: PCT/AU2022/051229

§ 371 Date: Apr. 11, 2024

(30) Foreign Application Priority Data
Oct. 13, 2021 (AU) 2021903289

(51) Int. Cl. C12N 5/076; A01N 1/02; A61D 19/02

(52) U.S. Cl. CPC C12N 5/061; A01N 1/0215; A01N 1/0226; A61D 19/02; C12N 2500/32; C12N 2500/34

(57) ABSTRACT

A sperm extender medium for prolonging the fertility of bovine spermatozoa during liquid storage of the spermatozoa. The medium includes a monosaccharide energy source for the spermatozoa. The one or more α or β amino acid osmolytes are selected from mono-amino carboxylic and mono-amino sulfonic acids, each independently having an R side group selected from the group consisting of H and non-polar, neutral open aliphatic chains excluding branched C3 and longer aliphatic chains. The medium also includes one or more antioxidants and physiologically acceptable inorganic salts for maintaining sperm viability, and is buffered for maintaining pH in a range of from about 7.0 to about 8.5. Further, the medium may have a concentration of sodium ions of less than about 95 mM, and an osmolarity in a range of from about 290 mOsm/L to about 400 mOsm/L. Preparations including the spermatozoa and methods for preparing the medium and methods for fertilisation of an ovum.

### FIELD OF THE INVENTION

The present invention relates to a sperm extender medium for extending the fertility of spermatozoa separated from seminal plasma. The invention also relates to the use of spermatozoa maintained in the extender medium for in vitro or in vivo fertilisation of ovum

### BACKGROUND TO THE INVENTION

The use of artificial insemination (AI) for the genetic improvement of beef cattle herds in northern Australia and other remote locations is restricted by logistical constraints of conducting artificial insemination (AI) programs on geographically isolated farms, the reduced fertility of cryopreserved spermatozoa and the limitations of oestrous synchronisation regimens. Besides the adverse effect of cryopreservation on post-thaw viability and fertility of spermatozoa, the use of cryopreserved spermatozoa further involves specialised storage and transport requirements, and reliance on liquid nitrogen.

Whilst the stallion semen extender INRA 96™ (imv Technologies Group), has previously been described as being optimal for the liquid storage of bull spermatozoa, fertility of bovine spermatozoa at room temperature has nevertheless only been maintained for up to approximately 3 days (Murphy, Murphy et al. 2017). This is far from adequate in many situations, limiting its application.

There remains a need for an extender medium for liquid storage of bovine spermatozoa and spermatozoa of other livestock animals such as sheep and goats.

### SUMMARY OF THE INVENTION

Broadly stated, the present disclosure relates to the provision of a sperm extender medium for liquid storage of the spermatozoa following separation from seminal plasma. The extender medium is therefore for use with fresh spermatozoa in the substantial absence of seminal plasma as distinct from a semen extender medium for addition to a semen sample such as for cryopreservation of the semen.

In arriving at the present invention the inventors confirmed that equine INRA 96™ extender medium was inadequate to maintain the fertility of bovine spermatozoa at ambient temperature for more than a limited period. The inventors also found that an optimised proprietary equine sperm extender medium their research group had developed over a period of 8 years was also not able to adequately maintain the viability and motility of bovine spermatozoa, indicating that bovine spermatozoa have specific requirements that are not met by sperm extender formulations developed for the horse. This result was unexpected as it had been thought by the inventors that their proprietary equine extender medium would have been suitable for adequately prolonging the fertility of bovine spermatozoa during liquid storage at ambient temperature.

As a result of further research, the inventors have now found that the use of certain amino acid(s) as osmolyte(s) in a sperm extender medium formulated for bovine spermatozoa can substantially prolong the fertility of fresh bovine spermatozoa at ambient/room temperature in the absence of cryopreservation of the spermatozoa. In particular, as described herein the inventors have shown that the fertility of bovine spermatozoa can be extended well beyond the 3 days at ambient temperature previously reported with the use of INRA 96™ with equal or superior fertility to cryopreserved spermatozoa. It is believed by the inventors that this is the first report of bovine spermatozoa having been stored in liquid form at ambient temperature without cryopreservation with retention of satisfactory fertilization potential of the spermatozoa for longer than 3 days.

However, the present disclosure is not limited to the use of the sperm extender medium only with bovine spermatozoa and the extender medium and extends to use with spermatozoa of other ruminant livestock animals such as domestic sheep and goats.

More particularly, in an aspect of the present invention there is provided a sperm extender medium for prolonging the fertility of bovine spermatozoa during liquid storage of the spermatozoa, the medium comprising one or more amino acid osmolytes in a nutrient solution for the spermatozoa and the one or more amino acid osmolytes being selected from mono-amino carboxylic and mono-amino sulfonic acids, each independently having an R side group selected from the group consisting of H and non-polar, neutral open aliphatic chains excluding branched C3 and longer aliphatic chains, wherein the medium is buffered for maintaining pH of the medium in a range of from about 7.0 to about 8.5, the medium having a concentration of sodium ions of less than about 95 mM, and an osmolarity in a range of from about 290 mOsm/L to about 400 mOsm/L.

Typically, the nutrient solution comprises one or more energy sources for the spermatozoa.

In at least some embodiments, the nutrient solution can also include one or more antioxidants.

In another aspect of the present invention there is provided a sperm extender medium for prolonging the fertility of spermatozoa of a livestock animal during liquid storage of the spermatozoa, the animal being selected from bovine, ovine and caprine animals, and the medium comprising:
a monosaccharide energy source for the spermatozoa;
one or more α or β amino acid osmolytes selected from mono-amino carboxylic and mono-amino sulfonic acids, each independently having an R side group selected from the group consisting of H and non-polar, neutral open aliphatic chains excluding branched C3 and longer aliphatic chains;
one or more antioxidants;
physiologically acceptable inorganic salts for maintaining sperm viability; and
wherein the medium is buffered for maintaining pH of the medium in a range of from about 7.0 to about 8.5, the medium having a concentration of sodium ions of less than about 95 mM, and an osmolarity in a range of from about 290 mOsm/L to about 400 mOsm/L.

Typically, in embodiments of the present disclosure each amino acid osmolyte is independently unsubstituted or is monosubstituted with a single R side group of other than H.

Typically, the R side group of each amino acid osmolyte in accordance with the present disclosure is independently either H, an aliphatic C1-C2 group, or an unbranched C3 chain.

When the R side group is a C2 chain, the C2 chain can be monosubstituted with a C1 group and typically, with a methyl group.

Typically, the R side group is independently either H or a C1-C2 alkyl.

Typically, each amino acid osmolyte independently has a formula selected from the group consisting of H₂N—CH₂—COOH, H₂N—CHR—COOH, H₂N—C₂H₃(R)—SO₂OH and H₂N—CH₂—CH₂—SO₂OH, wherein R is the R side group and is other than H.

In particularly preferred embodiments, each amino acid osmolyte is independently an amino acid of the formula H₂N—CH₂—COOH, H₂N—CH(R)—COOH or H₂N—CH₂—CH₂—SO₂OH, and most preferably an amino acid of the formula H₂N—CH₂—COOH or H₂N—CH(R)—COOH, wherein R is other than H.

Typically, an amino acid osmolyte used in a sperm extender medium in accordance with the invention will have an isoelectric point of from about 5.96 to about 6.0, the isoelectric point being the pH at which the amino acid has no net electrical charge.

In another aspect of the invention there is provided a sperm extender medium for prolonging the fertility of bovine spermatozoa of a livestock animal during liquid storage of the spermatozoa, the medium comprising one or more amino acid osmolytes in a nutrient solution for the spermatozoa, each said amino acid osmolyte independently having an isoelectric point in a range of from about 5.96 to about 6.0, wherein the medium is buffered for maintaining pH of the medium in a range of from about 7.0 to about 8.5, the medium having a concentration of sodium ions of less than about 95 mM, and an osmolarity in a range of from about 290 mOsm/L to about 400 mOsm/L.

Typically, the at least one amino acid osmolyte in a sperm extender medium embodied by the invention comprises at least the majority of α and/or β amino acid(s) in the extender medium in terms of concentration. In particularly preferred embodiments, the extender medium does not include any further α and/or β amino acid(s) beyond the one or more amino acid osmolytes.

In particularly preferred embodiments, the at least one amino acid osmolyte is selected from the group consisting of glycine, alanine, valine, taurine and combinations thereof.

In another aspect of the invention there is provided a sperm extender medium for prolonging the fertility of bovine spermatozoa during liquid storage of the spermatozoa, the medium comprising one or more amino acid osmolytes in a nutrient solution for the spermatozoa and the one or more amino acid osmolytes being selected from the group consisting of glycine, alanine, valine and taurine, wherein the medium is buffered for maintaining pH of the medium in a range of from about 7.0 to about 8.5, the medium having a concentration of sodium ions of less than about 95 mM, and an osmolarity in a range of from about 290 mOsm/L to about 400 mOsm/L.

Where stereoisomers of the one or more amino acid osmolyte(s) exist, the L-form of the amino acid will generally be used in the sperm extender medium.

Typically, the at least one amino acid osmolyte in a sperm extender medium in accordance with the invention is a glucogenic amino acid.

More preferably, the at least one amino acid osmolyte is selected from the group consisting of glycine, alanine, valine and combinations thereof.

Most typically, the at least one amino acid osmolyte is glycine.

Embodiments of the present disclosure may also comprise one or more agents selected from the group consisting of further organic osmolytes, antihyperglycemic agents for enhancing uptake of the monosaccharide energy source by the spermatozoa, sperm anti-agglutination agents, buffering agents, and antimicrobial agents.

Whilst the sperm extender medium described herein has use for extending the fertility of bovine spermatozoa during liquid storage at ambient temperature, in other embodiments, the sperm extender medium may be used for prolonging the fertility of spermatozoa of other ruminant livestock animals selected from sheep and goats.

Thus, in another aspect of the present disclosure there is provided a composition for a sperm extender medium for prolonging the fertility of spermatozoa of a livestock animal during liquid storage of spermatozoa in the medium as described herein, the animal being selected from bovine, ovine and caprine animals, and the composition comprising the one or more α or β amino acid osmolytes, the one or more antioxidants, and the physiologically acceptable inorganic salts for maintaining sperm viability, for addition to a predetermined volume of water to provide the sperm extender medium. The composition can also include other ingredient(s) of the extender medium as described in.

In another aspect there is provided a method for preparing a sperm extender medium for prolonging the fertility of spermatozoa of a livestock animal during liquid storage of the spermatozoa, the animal being selected from bovine, ovine and caprine animals, and the method comprising mixing one or more α or β amino acid osmolytes in a liquid medium in providing a sperm extender medium embodied by the present disclosure, the α or β amino acid osmolytes being selected from mono-amino carboxylic and mono-amino sulfonic acids, each independently having an R side group selected from the group consisting of H and non-polar, neutral, open aliphatic chains excluding branched C3 and longer aliphatic chains as described herein.

In another aspect there is provided a preparation of spermatozoa of a livestock animal in a sperm extender medium in accordance with the present disclosure.

Typically, semen of the animal is processed to separate the spermatozoa from substantially all seminal plasma prior to resuspension of the spermatozoa in the sperm extender medium in accordance with the present disclosure.

In at least some embodiments, the spermatozoa may be prepared by centrifuging the semen on a density gradient to separate the spermatozoa from seminal plasma, and resuspending the spermatozoa in a sperm extender medium in accordance with the present disclosure.

In another aspect of the present disclosure there is provided a method for fertilisation of an ovum of a livestock animal, comprising contacting the ovum with spermatozoa administered in a sperm extender medium as described herein, the spermatozoa being of the same animal type as the ovum. The method can comprise an in vitro or in vivo method of fertilisation (e.g., artificial insemination).

In at least some embodiments, the fertility of the spermatozoa as described herein may be retained in an extender medium in accordance with the present disclosure at ambient temperature for a period of greater than 3 days and typically, at least 5 days, at least 7 days or even at least 10 days in the absence of cryopreservation of the spermatozoa.

The livestock animal may be of any breed of domestic cattle (i.e., Bos taurus or e.g., Bos indicus), or can be a domestic sheep (Ovis aries) or goat (Capra hircus) which with cattle are members of the Bovidae animal sub-family.

The term “open aliphatic chain” as used herein in the context of an R side group of an amino acid osmolyte in accordance with the present disclosure is to be taken to mean a linear or branched carbon chain which does not form a ring or contain a cyclic or aryl ring group or structure such as in proline, tryptophan and phenylalanine, and to exclude carbon side groups (e.g., a C1-C3 group) substituted with a cyclic carbon or aryl group. For the purposes of describing the present invention, the term open aliphatic chain is also to be taken to exclude carbon C1-C3 groups which are substituted with, or coupled directly to, a sulfur atom or other heteroatom e.g., selected from oxygen, nitrogen or phosphorus (i.e., O, N or P), and so also excludes the sulfur containing R side groups of e.g., cysteine, homocysteine and methionine. As branched aliphatic C3 or longer (C4 etc) aliphatic chains are excluded from a bovine extender medium in accordance with the present disclosure, amino acids such as leucine and isoleucine are also excluded from use.

By prolonging the fertility of spermatozoa of the livestock animal at ambient temperature utilising a sperm extender medium as described herein, logistical obstacles to collecting and transporting sperm preparations to remote geographical locations in the absence of cryopreservation of the spermatozoa may be ameliorated. Moreover, by reducing or avoiding the need for cryopreservation in accordance with one or more embodiments of the present disclosure, not only may the spermatozoa retain fertility for a longer period at ambient temperature but also, the fertility of the spermatozoa may be superior to the corresponding cryopreserved spermatozoa.

By “prolonging the fertility” of spermatozoa as used herein is meant prolonging the viability and motility of the spermatozoa whereby the spermatozoa remain capable of fertilising ova of the same animal species.

The difference between the recitation of concentration in terms of “molarity” compared to “molality” in the context of the present disclosure is for all practical purposes negligible. Hence, concentrations described or defined herein in terms of “molarity” are to be considered to be the same in terms of “molality”. That is, for example, a concentration of “X mOsm/L” is to be considered for all purposes the same as “X mOsm/kg” of the relevant solution or medium.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers, integers or steps.

Any discussion of documents, acts, materials, devices, articles or the like that has been included in this specification is solely for the purpose of providing a context for the invention. It is not to be taken as an admission that any or all of these matters form part of the prior art base or were common general knowledge in the field relevant to the invention as it existed in Australia or elsewhere before the priority date of this application.

The features and advantages of the present disclosure will become further apparent from the following detailed description of non-limiting embodiments together with the accompanying drawings.

### BRIEF DESCRIPTION OF THE ACCOMPANYING DRAWINGS

FIG. 1: Total (A) and progressive (B) motilities of bovine spermatozoa (N=8) stored in a base bovine extender solution over 8 days at either room temperature (RT) or 17° C., with or without Nystatin for control of fungal growth.

FIG. 2: Total and progressive motilities (left) and viability and acrosome integrity (right) of bovine spermatozoa stored for 3 and 7 days in either INRA96™ at 5° C. and the base bovine extender solution at room temperature. *P≤0.05, **P≤0.01, ***P≤0.001.

FIG. 3: Total and progressive motilities of bovine spermatozoa stored in the base bovine extender medium supplemented with an osmolyte selected from soluble amino acids, NaCl or choline chloride after 7 days of storage at RT. Significant differences compared to the NaCl control. *P≤0.05, **P≤0.01 ***P≤0.001.

FIG. 4: Illustrates the use of a simple water jacket about a collection tube to prevent cold-shocking spermatozoa during bovine semen collection.

FIG. 5: Total and progressive motilities of bovine spermatozoa stored in base bovine extender solution supplemented with NaCl, choline chloride, glycine, valine or alanine after 3, 7, 10 or 14 days of storage at RT. Significant differences compared to the NaCl control. *P≤0.05, **P≤0.01 ***P≤0.001 ****P≤0.0001.

FIG. 6: Total and progressive motilities of spermatozoa stored in New bovine extender medium containing glycine (82.2 mM) for 7 and 14 days compared to spermatozoa cryopreserved in AndroMed™. Different superscripts denote a significant difference. Individual differences: NEW 7 over Cryo total motility P≤0.0001, progressive motility P≤0.05; NEW 7 over NEW 14 total motility P≤0.001, progressive motility P≤0.05. No significant difference between Cryo and NEW 14 for either parameter.

FIG. 7: Fertilisation rate (expressed as percentage of presumptive zygotes with two pronuclei; 2PN) following IVF using spermatozoa stored in NEW bovine extender medium containing glycine (82.2 mM) for 7 or 14 days compared to spermatozoa cryopreserved in AndroMed™. *** denotes P≤0.001.

### DETAILED DESCRIPTION OF EXEMPLARY EMBODIMENTS OF THE INVENTION

In arriving at the present invention the inventors have found that bovine spermatozoa have surprisingly different requirements for maintenance of fertility potential compared to stallion spermatozoa during liquid storage of the spermatozoa and that the fertility potential of spermatozoa such as from a bovine animal as described herein can be markedly prolonged in an extender medium by the use of some amino acid osmolytes compared to others.

Examples of amino acid osmolytes that can be utilised in a sperm extender medium embodied by the invention are glycine, alanine, valine and taurine. The first three of those amino acids are a mono-amino carboxylic acids whilst taurine (2-aminoethane-1-sulfonic acid) is an example of a suitable β mono-amino sulfonic acid as described herein. Whilst glycine and taurine are unsubstituted (and so the R side group of those amino acids is H), the R side group of alanine is methyl (a C1 group), whilst the R side group of valine is 1-methyl-ethyl (a substituted C2 chain). The hydrogen R group of glycine and the R side groups of each of alanine, valine and taurine are all non-polar, neutral side groups.

Unexpectedly, of the 7 common other α-amino acids classified as being non-polar with glycine, alanine and valine due to their non-polar R side groups i.e., leucine, isoleucine, proline, cysteine, methionine, phenylalanine and tryptophan, showed no or only relatively limited capability to prolong bovine sperm motility at room temperature. Similarly, comparatively poor results were found for the remaining 10 common α-amino acids classified as polar, basic, or acidic amino acids on the basis of their R side chain, as described in the Examples further below.

It is particularly preferred that each amino acid osmolyte selected for use in a sperm extender medium in accordance with the present disclosure is glucogenic. By “glucogenic” amino acid is meant an amino acid that can be converted into glucose, pyruvate or an intermediate in the tricarboxylic acid (TCA) cycle (also known as the Krebs cycle) and so can be utilised in the production of adenosine triphosphate (ATP) for providing cellular energy. Advantageously, a glucogenic amino acid may therefore not only act as osmolyte but may also act as a carbon energy source for generation of ATP, prolonging bovine sperm motility and so fertility.

The amino acid osmolyte(s) useful in a sperm extender medium in accordance with the present disclosure will typically independently have a single R side group selected from H, methyl and 1-methyl-ethyl. Whilst mixtures of two or more suitable physiologically acceptable amino acid osmolytes as described herein may be utilised, only a single such α or β amino acid osmolyte may be present in the medium.

Typically, the isoelectric point (pI) of an amino acid osmolyte useful in a sperm extender medium as described herein is between about 5.96 and about 6.0. The pI of glycine for instance is 5.97 whilst the pI for alanine is 6.0. The pI for valine is 5.96 This contrast with the other polar amino acids of isoleucine (pI=6.02), proline (pI=6.3), cysteine (pI=5.07), methionine (pI=5.74), phenylalanine (pI=5.48), and tryptophan (pI=5.89) (Lide D R, Handbook of Chemistry and Physics, 72^{nd} Edition, CRC Press, Boca Raton, FL, 1991).

Typically, in embodiments described herein, the total or combined overall concentration of the α- and/or β-amino acid osmolyte(s) in the medium as described herein will generally be in a range of from 40 mM to about 150 mM, more usually from about 70 mM to about 90 mM and typically, will be about 75 mM, 76 mM, 77 mM, 78 mM, 79 mM, 80 mM, 81 mM, 82 mM, 83 mM, 84 mM or about 85 mM.

The α and/or β amino acid osmolyte(s) will generally comprise greater than 50% of α and/or β amino acid(s) in the extender medium in terms of concentration and generally, greater than 55%, 60%, 65% or 70% and more usually, at least about 75% or more, 80% or more, 85% or more, 90% or more, or 95% or more of α and/or β amino acid(s) in the medium. Typically, the one or more amino acid osmolyte(s) will comprise 100% of α and/or β amino acid(s) in the extender medium.

In particularly preferred embodiments, one or more further organic osmolytes can be included in the extender medium and may, for example, be selected from L-carnitine, choline (typically as the chloride salt), myo-inositol, other α and β amino acid osmolytes, and combinations thereof. L-carnitine for instance is known to transport long-chain fatty acids into mitochondria of cells for energy production and is an antioxidant. In embodiments described herein the use of L-carnitine in combination with amino acid osmolyte(s) in the extender medium of the invention is particularly preferred. Typically, the overall concentration of further organic osmolyte(s) in a bovine extender medium in accordance with the invention will be the range of from about 50 mM to about 150 mM, more usually from about 80 to about 110 mM and preferably will be from about 85 mM to about 105 mM (e.g., 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, 100 mM, 101 mM, 102 mM, 103 mM, 104 mM or 105 mM).

Whilst any suitable monosaccharide energy source for bovine spermatozoa can be utilised in an extender medium in accordance with the present invention the monosaccharide energy source in at least some embodiments of the present disclosure may be selected from group consisting of fructose, glucose, galactose, mannose and combinations thereof. Typically, the monosaccharide(s) utilised will be present in the extender medium at a total concentration of from about 4 mM to about 10 mM, more usually about 5 mM to about 6 mM or 5.25 mM to about 5.75 mM and preferably, from about 5.0 mM to about 5.6 mM (e.g., 5.0 mM, 5.1 mM, 5.2 mM, 5.3 mM, 5.4 mM, or 5.5 mM, 5.6 mM, 5.7 mM, 5.8 mM, 5.9 mM or 6.0 mM).

The extender medium can also comprise one or more physiologically acceptable antihyperglycemic agents to enhance the uptake of the monosaccharide(s) by the spermatozoa. Rosiglitazone is a thiazolidinedione antihyperglycemic agent that for instance been reported to prolong fertility of equine spermatozoa at ambient temperature without cryopreservation, and is an activator of adenosine 5′-monophosphate protein kinase (AMPK) and Protein Kinase B (Atk) (Swegen et al. 2016; Ortiz-Rodriguez, 2019). Whilst rosiglitazone is a preferred antihyperglycemic agent that may be utilised in the bovine extender medium the invention is not limited thereto and other antihyperglycemic agents may be utilised, including any other suitable thiazolidinediones, or other such agents capable of activating AMPK and/or Akt. Typically, the antihyperglycemic agent(s) when used in the extender medium will be present at a concentration of from about 1 mM to about 60 mM, more usually from about 10 mM to about 50 mM, and more preferably in a range of from about 15 to 30 mM e.g., about 15 mM, 20 mM, 25 mM or 30 mM).

The extender medium in at least some forms may also include one or more organic carbon energy sources besides the monosaccharide(s). Examples of further such energy sources include pyruvate, which may be added in any physiologically acceptable salt form, such as sodium pyruvate. When present, the pyruvate will generally be employed at a concentration range of from about 0.5 mM to about 10 or 20 mM. More typically, pyruvate will be present in the extender medium at a concentration of from about 3 mM to about 8 mM, more usually from about 4.5 mM to about 6.5 mM and preferably from about 5 mM to about 6 mM (e.g., 5.0 mM, 5.1 mM, 5.2 mM, 5.3 mM, 5.4 mM, 5.5 mM, 5.6 mM, 5.7 mM, 5.8 mM, 5.9 mM or 6.0 mM).

The role of antioxidant(s) in embodiments of the extender medium of the invention is primarily to scavenge free radicals and particularly reactive oxygen species (ROS) such as peroxides, superoxides, hydroxyl radicals and singlet oxygen species. Suitable further antioxidant(s) which may be utilised in an extender medium embodied by the invention include coenzyme Q10. Other antioxidants which may be utilised include α-tocopherol, melatonin, and vitamin C. Such further anti-oxidants will typically be present at a total overall concentration of about 10 mM to about 175 mM, more usually from about 90 mM to about 150 mM and preferably, at a concentration of from about 100 mM to about 140 mM (e.g., 100 mM, 105 mM, 110 mM, 115 mM, 125 mM, 130 mM, 135 mM or 140 mM).

Another carbon energy source for the spermatozoa that can be included in in a sperm extender medium as described herein is lactate. Lactate can also assist to maintain pH by acting as an acidity regulator. Typically, when used, lactate may be present in the extender medium at a concentration in a range of from about 10 mM to about 30 mM, and more usually in a range of from about 15 mM to about 25 mM (e.g., 15 mM, 16 mM, 17 mM, 18 mM, 19 mM, 20 mM, 21 mM, 22 mM, 23 mM, 24 mM or 25 mM).

Other acidity regulators that can be utilised in the extender medium include bicarbonate (e.g., at a concentration of from about 5 mM to about 45 mM, more usually in range of from about 10 mM to about 40 mM or 15 to 35 mM and preferably, a concentration in a range about 20 mM to about 30 mM), typically utilised as the sodium salt.

Buffering agents which may be utilised include HEPES (4-(2-hydroxyethyl)-1-piperazineethansulfonic acid) (e.g., Gibco) and will normally be employed at a concentration of from about 1-5 mM and more usually, from about 1 mM to about 3.0 mM and most preferably, at a concentration of about 2 mM. Whilst HEPES is preferably used to buffer pH of bovine sperm extender medium in accordance with the present invention, other suitable conventional physiologically acceptable zwitterionic buffering agents or systems may also be employed.

The sperm extender medium as described herein can further comprise agents as may be selected from sperm anti-agglutination agents such as polyvinyl alcohol (PVA), anti-microbial agents such as penicillin, streptomycin, gentamicin and amikacin, and anti-fungal agents such as nystatin and fluconazole.

Physiologically acceptable inorganic salts for maintaining sperm viability in the extender medium will typically be selected from inorganic salts of potassium, calcium, magnesium and sodium, such as chloride, phosphate and sulfate salts thereof. Particular salts that can be included in the extender medium include KCl, CaCl₂), KH₂PO₄, MgSO₄ and NaCl. Such salts are commonly used in cell and embryo culture medium to provide K⁺, Na⁺, Ca2⁺, Mg2⁺, Cl⁻, SO4²⁻ and/or PO₄²⁻ ions in solution for e.g., maintaining cell membrane potential and for providing cell nutrient requirements. Typically, the concentrations of respective of the inorganic salts in the bovine extender medium will be in a range of from about 1 mM to about 5 mM. Magnesium though is found in semen in relatively higher amounts and will typically be provided in at least some embodiments of the bovine sperm extender medium of the invention (e.g., provided as MgSO₄) in a concentration in a range of from about 55 mM to about 65 mM (e.g., 55 mM, 56 mM, 57 mM, 58 mM, 59 mM, 60 mM, 61 mM, 62 mM, 63 mM, 64 mM, or 65 mM).

However, as described further below, sodium ion concentration has been reported to be deleterious to sperm longevity in vitro believed by the inventors to be due to depletion of cellular adenosine triphosphate (ATP) levels by activity of Na⁺/K⁺ ATPases for maintenance of cell homeostasis. In embodiments of the invention the concentration of sodium ions in the bovine extender medium is desirably minimised to a level of less than about 95 mM, 90 mM, 85 mM, or 80 mM, more usually to a level of about 70 mM or less, 65 mM or less, 60 mM or less and most preferably, to a level of about 50 mM or less.

Typically, the medium is buffered within a pH range of from about 7.0 to about 8.5 by the buffering system formed by the combination of buffering agent(s) and acidity regulator(s) utilised in the extender medium, and more preferably, from about 7.5 to about 8.5. The pH of the medium can be adjusted to within this range by use of suitable acid or base such as HCl or KOH although the final pH of the medium is determined by the particular amino acid osmolyte(s) utilised.

The osmolarity of the sperm extender medium will generally be in a range of from about 290 mOsm/L to about 400 mOsm/L and more usually, in a range of from about 290 mOsm/L to about 390 mOsm/L, 380 mOsm/L, 370 mOsm/L, 360 mOsm/L, 350 mOsm/L or 340 mOsm/L, or from about 300 mOsm/L to about 340 mOsm/L e.g., 300 mOsm/L, 305 mOsm/L, 310 mOsm/L, 315 mOsm/L, 320 mOsm/L, 325 mOsm/L, 330 mOsm/L, 335 mOsm/L or 340 mOsm/L.

Whilst particular concentration ranges of components of a sperm extender medium in accordance with the present disclosure have been described above, it will be understood that all concentrations of the relevant component with the upper and lower limits of the described concentration range are expressly provided for herein.

As described herein, a sperm extender medium embodied by the present disclosure may be used for prolonging fertility of spermatozoa of the livestock animal at ambient temperature in the absence of cryopreservation pending use of the spermatozoa for in vitro or in vivo fertilisation employing conventionally known such techniques. In particularly preferred embodiments, the sperm extender medium may maintain fertility of the spermatozoa of the livestock animal in liquid storage at ambient temperature for a period of more than 3 days, and in preferred embodiments for at least 5 days, in the absence of cryopreservation of the spermatozoa.

Typically, the semen will be collected into a physiologically acceptable medium in a suitable collection receptacle (e.g., a commercially available conical, inert plastic centrifugation tube) or otherwise be collected in a suitable such receptacle and be diluted with the physiologically acceptable medium of choice (e.g., a suitable cell culture or an equine or bovine sperm extender medium) prior to centrifugation to separate the spermatozoa from the seminal plasma preferably via a density gradient centrifugation protocol, resuspension of the separated spermatozoa and washing of the spermatozoa one or more times via further centrifugation, and final resuspension in a sperm extender medium embodied by the invention for liquid storage at ambient temperature until use. Desirably, the physiologically acceptable medium into which the bovine serum is collected is pre-warmed if required to an appropriate temperature e.g., in a range of from about 25° C. to about 37° C. (e.g., 30° C.). The medium used for the washing step(s) can likewise be pre-warmed. In particularly preferred embodiments, a sperm extender medium in accordance with the present invention is used as the physiologically acceptable medium into which the semen is collected as well as for the washing step(s).

The ambient or room temperature (RT) at which the spermatozoa is stored until use desirably ranges from e.g., 17° C. to about 25° C. and is preferably about 22° C. The spermatozoa can for instance be stored in air-conditioned rooms or environments, or in insulated carriers cooled by conventional cold, gel or ice packs as commonly known until use.

A sperm extender formulation as described herein can be provided in solid (e.g., as a powder) or in part solid form for being added or mixed together to form an extender medium in accordance with the invention. For example, some components of the extender medium may be provided in solution and others in powder form for being mixed together to final volume in e.g., double-distilled (ddH₂O) or deionised water and predetermined concentrations of the respective components of the medium. Further, some embodiments of a sperm extender medium in accordance with the invention may include components such as coenzyme Q10 and rosiglitazone which have low aqueous solubility and may require pre-solubilisation using a suitable solvent and/or heat. Coenzyme Q10 for example be dissolved in dimethylformamide at 42° C. whilst rosiglitazone can be dissolved in dimethyl sulfoxide (DMSO) at the same temperature to provide stock solutions for subsequent use. Additionally, the components of the extender formulation can be lyophilised and reconstituted with ddH₂O or deionised water provided separately for on-site use.

Whilst the invention is further described below with reference to a number of non-limiting examples of a sperm extender medium the present disclosure is not limited thereto and embodiments in accordance with the present disclosure may be used for prolonging the viability of spermatozoa of other ruminant livestock animal species in liquid storage, such as spermatozoa of domestic sheep or goats.

### Example 1: Optimisation of Semen Extender Medium

### 1. General Methodology

Equine sperm extender medium INRA96 was purchased from Minitube Australia (Ballarat, Victoria, Australia), and BoviPure™ (Nidacon International), used to isolate spermatozoa in Experiment 1 (see Section 2 below), was purchased from Tek Event (Round Corner, NSW, Australia). Unless otherwise stated, all other chemicals and reagents were purchased from Sigma-Aldrich (Castle Hill, NSW, Australia).

### 1.1 Medium BWW

A modified Biggers, Whitten and Whittingham (BWW) medium (Biggers, Whitten et al. 1971) containing 95 mM NaCl, 4.7 mM KCl, 1.7 mM CaCl2·2H2O, 1.2 mM KH2PO4, 1.2 mM MgSO4.7H2O, 25 mM NaHCO₃, 5.6 mM D-Glucose, 275 μM sodium pyruvate, 3.7 μl/ml 60% sodium lactate syrup, 50 U/ml penicillin, 50 μg/ml streptomycin, 0.25 mg/ml gentamicin to prevent growth of Pseudomonas aeruginosa (Aurich and Spergser 2007), 20 mM HEPES and 0.1% (w/v) polyvinyl alcohol, with an osmolarity of approx. 310 mOsm/L was utilized as the control medium throughout this study.

### 1.2 Gradient Centrifugation

Selection of high quality bull spermatozoa was achieved using a 45% and 90% discontinuous Percoll™ (GE Healthcare, Castle Hill, Australia) or BoviPure™ centrifugation gradient. For this procedure, Percoll™ (90 mL) was supplemented with 10 mL of Ham's F10 solution, 370 μL sodium lactate syrup, 3 mg sodium pyruvate, 210 mg sodium hydrogen carbonate and 100 mg polyvinyl alcohol (PVA). This isotonic Percoll™ solution or BoviPure™ was diluted with BWW to create the discontinuous gradient (45% and 90% isotonic Percoll™/BoviPure™: 55% and 10% BWW respectively). 3 mL of the 90% solution was underlaid below 3 mL of the 45% solution in 15 mL conical centrifuge tubes (BD Falcon).

### 1.3 Collection and Preparation of Spermatozoa

Institutional and New South Wales State Government ethical approval was secured for the use of animal material in this study. This research was based on ejaculates from 17 normozoospermic Angus bulls (between 3 and 7 years of age) of proven fertility, held on institutionally-approved premises at Charles Sturt University (Wagga Wagga, NSW, Australia). Semen was collected via electro-ejaculation, and the ejaculate was either left undiluted (Experiment 1) or was immediately diluted (2:1; extender:semen) with INRA96 (Experiments 2 and 3—Sections 2 and 3). This initial dilution was found to be beneficial to reduce the damage to spermatozoa caused by toxic seminal plasma proteins during processing (Bergeron and Manjunath 2006). Equipment and extender medium were maintained at temperatures between 30° C. and 37° C. for the duration of semen collection and dilution. The tubes of extended semen were then held in the laboratory at RT (approx. 20 to 25° C.) for up to 30 min prior to further processing. For Experiment 1, up to 6 mL of the extended semen was either centrifuged directly (700×g for 10 min), or was overlaid on top of BoviPure™ gradient and centrifuged at 700×g for 20 min, and for Experiments 2 and 3 up to 5 mL extended semen was layered on top of Percoll™ gradients and centrifuged at 700×g for 30 min. Following centrifugation, the seminal plasma and density gradient media were removed and discarded. Spermatozoa were recovered from the base of the tubes, resuspended in 2 mL BWW and washed via centrifugation (500×g for 5 min) with BWW. These pelleted cells were then resuspended at a concentration of 50×10⁶ spermatozoa/mL in the experimental extender media.

### 1.4 Sperm Motility Analyses

Sperm motility was objectively determined with computer assisted sperm analysis (CASA; IVOS, Hamilton Thorne, Danvers, MA) using the following settings; negative phase-contrast optics, recording rate of 60 frames/s, minimum contrast of 70, minimum cell size of 4 pixels, low size gate of 0.17, high size gate of 2.9, low intensity gate of 0.6, high intensity gate of 1.74, non-motile head size of 10 pixels, non-motile head intensity of 135, progressive VAP threshold of 50 μm/s, slow (static) cells VAP threshold of 20 μm/s, slow (static) cells VSL threshold of 0 μm/s and threshold STR of 75%. Cells exhibiting a VAP of ≥50 μm/s and a STR of ≥75 were considered progressive. Cells with a VAP greater than that of the mean VAP of progressive cells were considered ‘rapid’. A minimum of 200 spermatozoa in a minimum of five fields were assessed using 20 μm Leja standard count slides (Gytech, Australia) and a stage temperature of 37° C.

### 1.5 Flow Cytometric Viability and Acrosome Integrity Analyses

100 μL aliquots of sperm samples were pelleted via centrifugation (300×g for 3 min), resuspended in 200 μL BWW containing FITC-PNA (0.8 μg/mL) and LIVE/DEAD far red fixable stain (0.5 μL/mL; Molecular Probes, Australia), and incubated at 37° C. for 20 min. Following staining, spermatozoa were pelleted via centrifugation and re-suspended in BWW for flow cytometric analysis. All flow cytometry was performed using a FACSCanto flow cytometer (BD Bioscience, NJ, USA) with a 488 nm argon-ion laser. Emission measurements were made using 530/30 band pass (green/FL-1) and >670 long pass (far red/FL-4) filters. Debris was gated out using a forward scatter/side scatter dot plot, and a minimum of 10000 cells were analysed per sample. All data was analysed using FACSDiva software (BD Bioscience). Spermatozoa were classified as being either; live and acrosome intact (neither green nor red fluorescence), live and acrosome damaged (green fluorescence only), dead and acrosome intact (red fluorescence only), or dead and acrosome damaged (red and green fluorescence).

### 1.5 Statistical Analysis

All data used in this study were tested for normal distribution prior to analyses. If data could not be transformed to fit a normal distribution, a non-parametric test was performed. Data for all experiments were analysed by one way ANOVA with JMP, Version 14.0 software (SAS Institute Inc., Cary, NC). Where significant treatment effects were identified by ANOVA (α=0.05), means comparisons were performed. Differences between the parameters of spermatozoa stored in various treatment media in all experiments were identified using Student's t-tests for pair-wise mean comparisons (α=0.05).

### 2. Experiment 1—Optimisation of Sperm Preparation Protocols and Testing of a Proprietary Stallion Extender Medium on Bull Spermatozoa

The aim of this study was to conduct a preliminary investigation to ascertain whether bovine spermatozoa could be effectively stored at room temperature (RT) in a proprietary ambient temperature stallion sperm extender which has been developed by the inventor's research group over a period of 8 years (referred to below as the “proprietary” equine extender medium), and whether the isolation of high-quality spermatozoa from dead cells, seminal plasma and microbial contaminants using a BoviPure™ centrifugation step would be beneficial to sperm longevity during storage at RT. INRA96™ is a commercially available stallion semen extender which had previously been reported to be optimal for the liquid storage of bovine spermatozoa (Murphy, Murphy et al. 2017), was utilised as the control medium for this study.

Spermatozoa from 6 bulls were processed as outlined above, and CASA motility analyses were performed after 3 days of liquid storage at 17° C.

No statistical interaction between centrifugation treatment and sperm storage medium were observed, so data were pooled for further analyses. The progressive and total motilities of spermatozoa stored in INRA96™ were significantly higher than those stored in the proprietary equine extender medium (71 vs 31% and 63.3 vs 27% for total and progressive motilities respectively; P≤0.0001) and the isolation of spermatozoa using BoviPure™ resulted in significantly improved total and progressive motilities (59.1 vs 43.5% and 52.2 vs 38.1%, respectively; P≤0.01) compared to direct centrifugation alone.

These results show that bovine spermatozoa clearly have unique requirements during in vitro storage compared to equine spermatozoa. Additionally, the inventors observed that the isolation of bovine spermatozoa using density gradient centrifugation significantly improved sperm quality during storage. While others have reported that processing bovine spermatozoa through BoviPure™ and Percoll™ gradients does in fact improve sperm quality (Arias, Andara et al. 2017; Samardzija, Karadjole et al. 2006a; Samardzija, Karadjole et al. 2006b), there are no studies showing the long-term effects of density gradient centrifugation on spermatozoa intended for liquid storage. This step not only removes the dead and dying cells which contribute to oxidative stress in the remaining viable cells (Aitken, Naumovski et al. 2015; Upreti, Jensen et al. 1998), but it also significantly reduces the microbial and viral contaminants that may be present in the raw ejaculate (Galuppo, Junior et al. 2013; Varela, Rey et al. 2018)—a step which is important when spermatozoa is to be stored at higher temperatures.

### 3. Experiment 2—Determination of Extender Medium Requirements for Liquid Storage of Bovine Spermatozoa

Based upon the results of Experiment 1 above, in which the motility of bull spermatozoa stored in the proprietary equine extender formula were significantly poorer than those stored in INRA96™ equine semen extender, studies were undertaken to investigate the effects of different components on bovine sperm motility after 24 hours at RT.

Effects of common cell media parameters and ingredients including pH, sodium bicarbonate content, sodium pyruvate dose and osmolyte source (e.g., NaCl, L-carnitine, choline chloride) amongst others were observed during this study.

A pH of 7.5 to 8.5 was found to be preferred for both total and progressive motility. Sodium pyruvate concentrations of 0 mM, 0.275 mM, 5 mM, 10 mM and 20 mM were assessed. No significant difference in sperm motility was observed between the absence of sodium pyruvate and each of the test concentrations of 0.275 mM to 20 mM evaluated.

While NaCl is the most commonly utilised osmolyte in culture media, the inventors have recently found that alternative osmolytes such as L-carnitine or choline chloride may better preserve the longevity of spermatozoa during liquid storage at RT. In particular, in the present study, the inventors found that when 50% of the NaCl was replaced with either L-carnitine (100 mM) or choline chloride (95 mM), both total and progressive motilities of bovine spermatozoa were improved.

No significant effects of rosiglitazone dose at the concentrations evaluated (0-100 μM), sugar source (e.g., glucose, fructose, sucrose or raffinose) or coenzyme Q10 were recorded under field conditions at the 24 h mark as determined by CASA. However, test samples were subjectively monitored over successive days and treatments exhibiting the greatest sperm longevity (effectively those which took the longest for all cells to become immotile as assessed under light microscopy) were selected for inclusion in a base bovine extender solution considered by the inventors to be suitable for use in a base bovine extender solution. The components of the base solution are listed in Table 1 below.

TABLE 1
Bovine base extender solution

| Ingredient | Final concentration/volume | |
|---|---|---|
| Fructose | 5.55 | mM |
| pH | 7.9 | |
| Sodium Pyruvate | 5 | mM |
| Rosiglitazone | 50 | mM |
| NaCl | 41.1 | mM |
| L-Carnitine | 100 | mM |
| Sodium Bicarbonate | 25 | mM |
| Coenzyme Q10 | 40 | mM |
| KCl | 4.58 | mM |
| Cacl2, 2H2O | 1.63 | mM |
| KH2PO4 | 1.14 | mM |
| MgSO4, 7H2O | 59.64 | mM |
| Poly-vinyl Alcohol (PVA) | 11.6 | mM |
| HEPES | 2 | mM |
| Sodium Lactate | 19.8 | mM |
| Pen/Strep (5000 U Penicillin and | 10 | μL/mL |
| 5 mg/ml Streptomycin solution) | | |
| Gentamicin (50 mg/mL) | 5 | μL/mL |

4. Experiment 3—Comparison of the Base Bovine Extender Solution with INRA96

The base bovine extender solution was tested against chilled (5° C.) spermatozoa stored in INRA96™. Additional treatments for the base solution were storage of spermatozoa in the medium at either 17° C. or at RT (20-25° C.) and supplementation with or without Nystatin (100 U/mL) to inhibit fungal growth that was observed sporadically during Experiments 1 and 2 described above. Spermatozoa from a total of 8 bulls were processed as described above, with CASA motility measurements being performed every 24 h over 8 days, and chilled spermatozoa being assessed at 3 and 7 days. Acrosome integrity analyses were performed at 3 days for all samples, and at 7 days for the optimised bull spermatozoa extender treatments.

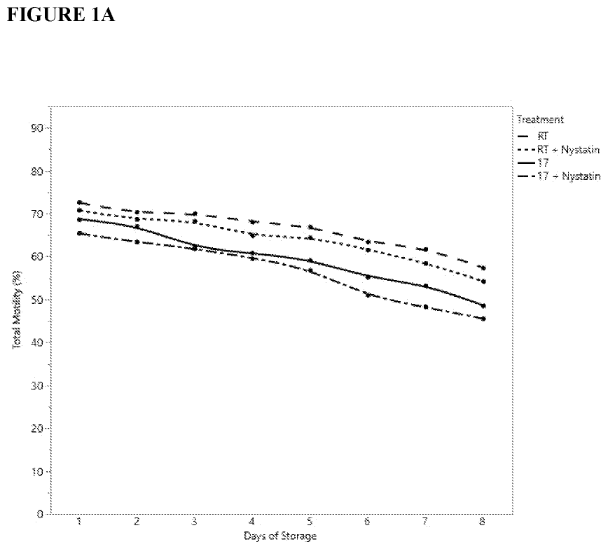

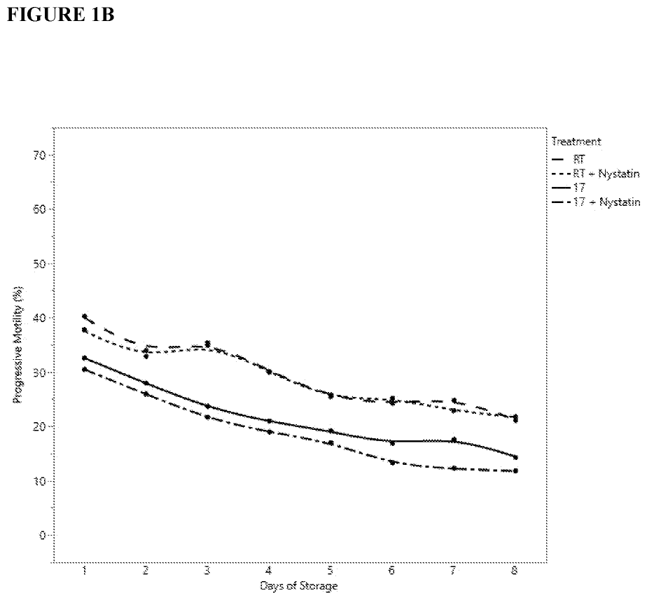

The total and progressive motility of bull spermatozoa stored in the base bovine extender medium at RT did not significantly decrease over a week of storage (FIG. 1). While the RT storage temperature (without Nystatin) produced consistently higher results than any other treatment, these were only significant at day 3 of storage, where the progressive motilities of both the RT and the RT+Nystatin treatment were observed to be significantly higher than both of the 17° C. treatments.

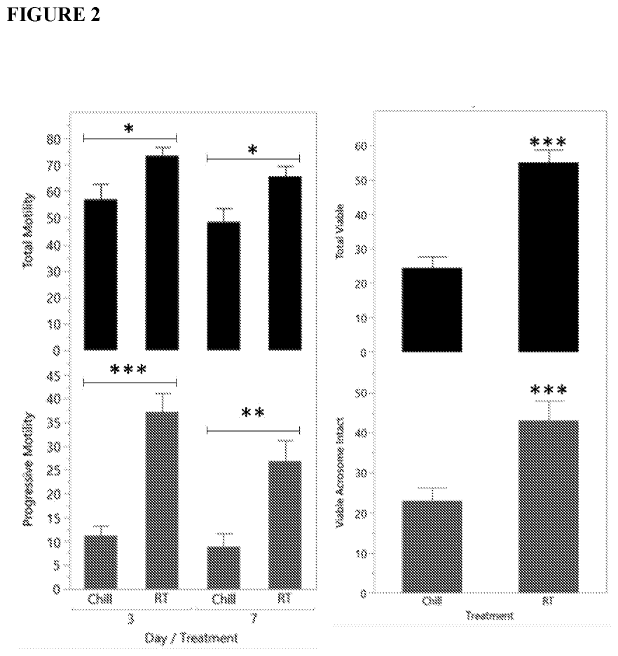

When compared with chilled spermatozoa stored in INRA96™, after 3 and 7 days of storage the total and progressive motilities were significantly higher in the base bovine extender solution. A similar pattern was observed for acrosome integrity and viability; after 3 days of storage (considered to be the maximum for sperm fertility following chilling), spermatozoa stored in the base bovine extender solution had significantly higher viability and acrosome integrity that chilled spermatozoa stored in INRA96™ (FIG. 2).

This is believed to be the first study to demonstrate the successful liquid storage of bovine spermatozoa at room temperature, and the first study to perform in vitro assessments of chilled bovine spermatozoa stored in INRA96™ for longer than three days (Murphy, Eivers et al. 2018).

The fact that there was no significant decline in the motility of spermatozoa stored in the base bovine extender medium over a full week of storage at room temperature suggests that the fertility of these cells is maintained and suitable for use in artificial insemination (AI) programs.

### Example 2: Development of Bovine Extender Medium and Fertility Studies

### 1. Semen Collection and Processing

Bull semen (animals were housed at Charles Sturt University, Wagga Wagga, NSW, Australia) was collected via electroejaculation, immediately diluted 2:1 (extender:semen) with equine INRA96™ semen extender, and maintained at room temperature (RT) before further processing. Extended semen was then layered onto a discontinuous (45/90%) Percoll™ gradient and centrifuged at 700×g for 30 min to isolate high quality spermatozoa to be used for storage experiments described below. The high-quality pelleted spermatozoa were then resuspended in modified Biggers, Whitter and Whittingham (BWW) medium (Gibb et al., 2014) and washed again via centrifugation (300×g for 5 min). Pelleted spermatozoa were then resuspended in the various formulations described in each experiment, and stored in 5 mL Sarstedt specimen pots at RT (21-23° C.) in the dark until they were assessed.

### 2. Comparison of Different Amino Acids on Bovine Sperm Motility

Sodium chloride has been shown to be detrimental to mammalian astrocyte longevity in vitro due to the use of Na⁺K⁺-ATPases for homeostasis (Silver and Erecińska, 1997), and the removal of NaCl in stallion sperm storage medium can significantly improve cell longevity via improved energy (ATP) retention (Gibb et al., 2015, Gibb et al., 2016). The inventors have also previously shown that replacement of NaCl with organic osmolytes such as L-carnitine can be beneficial during the storage of both stallion (Gibb et al., 2015, Gibb et al., 2016) and bull (Klein et al., 2019) spermatozoa. In leading to the present invention, a study was undertaken by the inventors to screen the suitability of soluble amino acids for use as osmolyte(s) in bovine extender medium.

This study was undertaken in 2 parts: the first was a screening of soluble amino acids (spermatozoa from split ejaculates of n=10 bulls), and the second was to confirm the results of the first part using only the most successful 3 amino acids (spermatozoa from split ejaculates of n=9 bulls). Sodium chloride and choline chloride were used as osmolyte controls. Choline chloride is a non-metabolically active osmolyte which does not exacerbate ATP loss via function of the aforementioned sodium pumps (Gibb et al., 2015).

The base bovine extender solution described above (Example 1, Experiment 3) was utilised for the purposes of these evaluations, with the exception that the 41.1 mM NaCl was not included in the base solution to which either the test amino acid or choline chlorine was added. Test amino acid was added to the base solution to a final concentration of 82.2 mM whilst the controls contained either the NaCl or choline chloride to a final concentration of 41.1 mM. The initial pH of solutions varied between 7-10 and were adjusted to be between 7.5 and 8.0 with either KOH or HCl. All test media were supplemented with 100 U/ml Nystatin to control fungal growth. Sperm motility was assessed as per Example 1.

For Part 1 of the study, motility was evaluated following 7 days liquid storage at room temperature (RT). Motility of spermatozoa was evaluated at 3, 7, 10 and 14 days liquid storage at RT for Part 2 of the study. Data were again analysed using JMP v14 software (ANOVA).

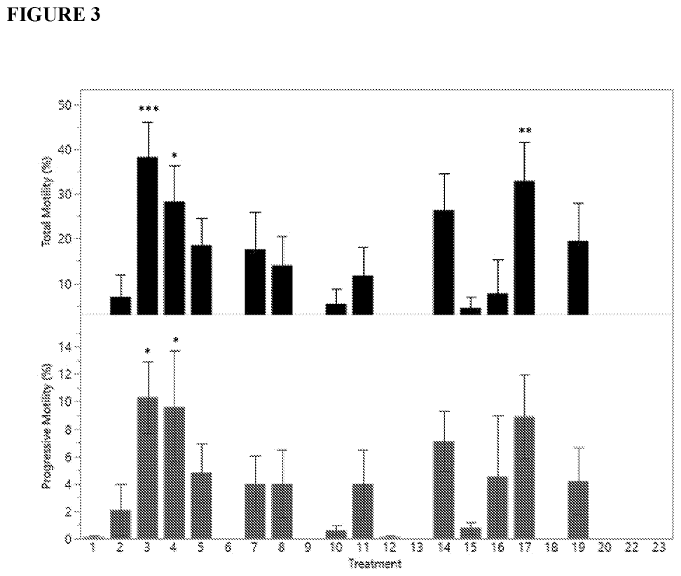

The results for the initial evaluation of the effect of different osmolytes on bovine sperm motility in Part 1 of the study are shown in the graph of FIG. 3. The identities of the respective test agents by column number in the graph are as follows: 1. asparagine, 2. methionine, 3. glycine, 4. taurine, 5. threonine, 6. leucine, 7. lysine, 8. proline, 9. cysteine, 10. arginine, 11. serine, 12. choline chloride, 13. tryptophan, 14. valine, 15. NaCl, 16. histidine, 17. alanine, 18. phenylalanine, 19. glutamine, 20. aspartic acid, 21. tyrosine, 22. glutamic acid, and 23. isoleucine. The amino acids utilised were all L-amino acids other than taurine and glycine which do not have D and L forms.

From FIG. 3, it can be seen that the total and progressive motilities of spermatozoa stored in media osmotically balanced using either glycine (treatment #3), taurine (treatment #4) or alanine (treatment #17) were significantly higher than all other treatments, including both the NaCl (treatment #12) and choline chloride (treatment #15) controls. It can also be seen that spermatozoa stored in the test media containing valine (treatment #14) also exhibited substantially better total and progressive motilities than the remainder of the treatments. Whilst bovine spermatozoa exhibit better total and progressive motility in extender base solution containing taurine at day 3 of liquid storage, spermatozoa were found by the inventors to have better motility in solution containing valine than taurine at day 14 (date not shown).

The overall low motility in all groups was believed to be due to cold shock of the spermatozoa at the time of collection. Part 2 of this study was undertaken following optimisation of semen collection conditions (see section 3 below), again employing NaCl and choline chloride as controls.

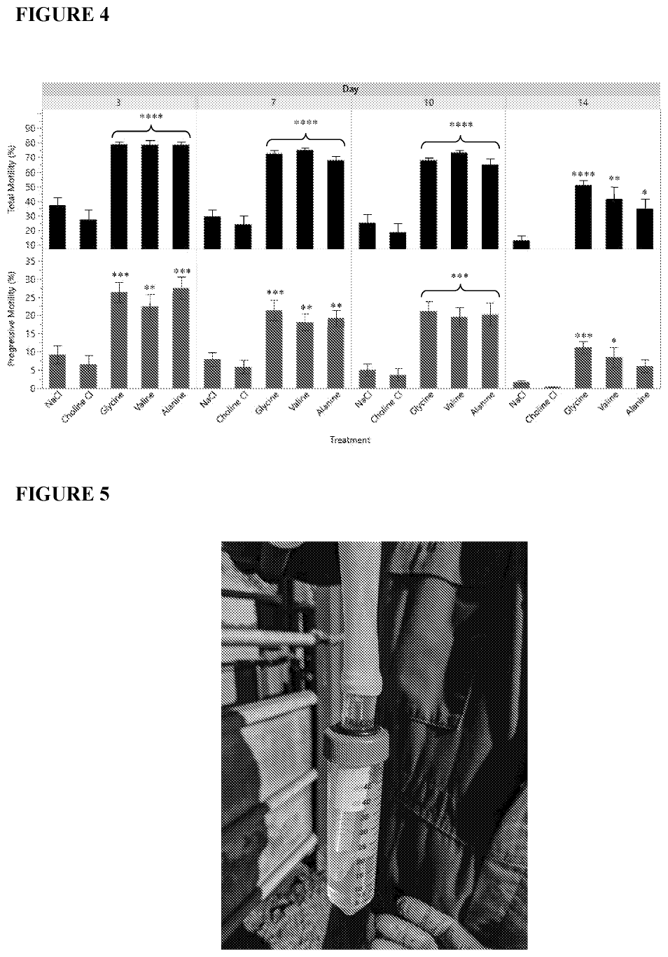

When the bovine base extender solution was supplemented with either glycine, valine or alanine as described above, the spermatozoa did not suffer any significant loss in motility between 3 and 10 days of storage at RT, though by day 14, motility had significantly declined. The motility of spermatozoa subjected to either glycine, valine or alanine was significantly higher than both of the controls at all time points. By day 14, the differences between the total and progressive motilities of spermatozoa stored in the glycine-containing media compared to the NaCl control were more highly significant than the other two amino acid treatments (FIG. 4). Glycine was therefore considered a preferred osmolyte for bovine spermatozoa compared to each of valine and alanine.

Extender medium comprising the base extender solution supplemented with either glycine (at 82.2 mM) without added NaCl (i.e., excluding the 41.1 mM concentration of NaCl in the formulation set out in Table 1) is referred to below as “New bovine extender medium”.

### 3. Optimisation of Semen Collection Conditions

As described immediately above, it became apparent that low ambient temperature was affecting the quality of the spermatozoa, resulting in low motilities across all treatments. For this reason, for part 2 of the experiment (conducted 2 weeks after part 1) additional steps were taken to ensure samples were kept warm, including the collection of semen into pre-warmed tubes inside water jackets filled with 37° C. water as generally illustrated in FIG. 5.

### 4. Fungicide Comparison

The storage of semen samples at room temperature is complicated by microbial contamination which is not curbed by thermal restriction as is the case for chilling and cryopreservation. Whilst Nystatin has been used in equine sperm extender medium to impede fungal growth, it has been suggested that this fungicide may be deleterious to bull spermatozoa (Foote, 2002). A more recent study has shown that the antifungal Fluconazole was both effective at controlling yeast contamination while having no detrimental effects on ram spermatozoa (Othman et al., 2016). Therefore, the effect of the previously used dose of Nystatin (100 U/mL) on bovine sperm motility was compared along with two doses of Fluconazole (12.5 mg/L and 25 mg/L) against a control with no antifungal.

Semen was collected, processed and stored according as above (Example 2, Section 1.1). Following 1, 3, 7, 10 and 14 days of storage at RT in the base extender solution as described (Example 2, Experiment 2) supplemented with glycine to a final concentration of 82.2 mM (pH of the medium was 7.9), sperm motility was assessed using CASA as above. Data were analysed using JMP v14 software (ANOVA).

No significant differences were observed between any treatments. As it was not detrimental to storage and is the more effective inhibitor of growth (Othman et al., 2016), Fluconazole (at 25 mg/L) was utilised in the extender formulation for the remaining experiments.

### Example 3: Motility and Fertility of Bull Spermatozoa Stored in New Bovine Extender Medium Vs Cryopreserved Spermatozoa

A study was conducted to compare the functionality and fertility of bovine spermatozoa stored in the bovine extender base solution (Example 2, Experiment 2) supplemented with glycine to a final concentration of 82.2 mM (and containing no included NaCl) for 7 and 14 days with spermatozoa cryopreserved using a standard commercial protocol in AndroMed™ semen extender (Minitube Australia Pty Ltd, Smythesdale, Victoria, Australia). Spermatozoa were collected and processed (Example 2, Section 1) before being split into 2 aliquots and either cryopreserved, or stored in the New bovine extender medium at RT (22° C. in a polystyrene box).

Following storage for 7 and 14 days, spermatozoa were analysed for motility (as per Example 1) and were utilised for IVF. For the IVF study, abattoir derived oocytes were matured in tissue culture medium 199 (TCM-199)+bovine serum albumin (BSA) for 22-24 hours, denuded and fertilised. Fertility was measured using the 2 pronuclei rate (2PN) at 18-20 hours following the addition of spermatozoa. Presumptive zygotes were fixed with 4% PFA, permeabilised with 0.3% Triton-X, stained with DAPI, and assessed using fluorescent microscopy. Motility and fertilisation data were analysed using JMP v 14 software and ANOVA.

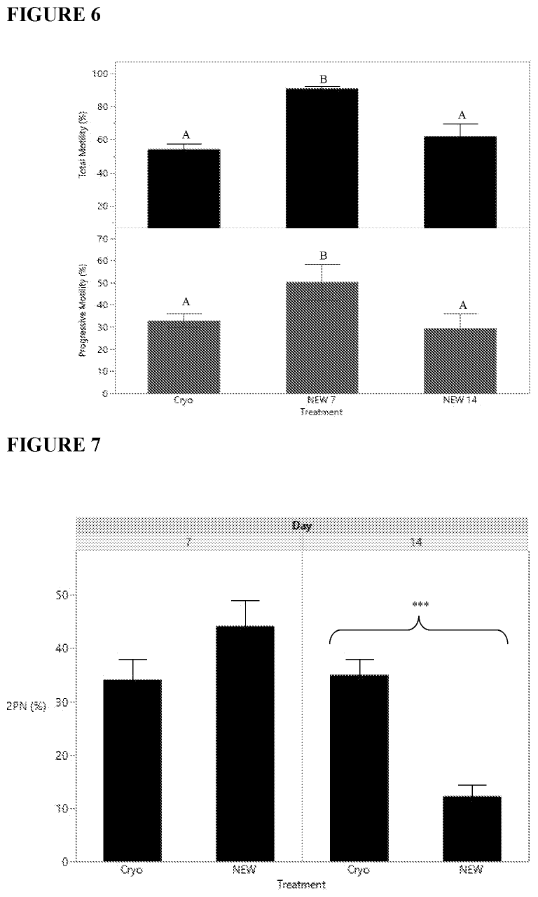

After 7 days of storage, the total and progressive motilities of spermatozoa stored in the New bovine sperm extender medium containing glycine were significantly higher than those of cryopreserved spermatozoa (P≤0.0001 and 0.05 for total and progressive motilities respectively). By 14 days of storage, the motilities of spermatozoa stored in New bovine extender medium had significantly declined, though they were still akin to those of cryopreserved spermatozoa (FIG. 6). However, the New bovine extender medium was found to support sperm longevity and fertility for at least 10 days.

In a similar pattern, in vitro fertilisation rates were higher for spermatozoa stored for 7 days in the New extender medium (44 vs 34% fertilised oocytes) compared to the cryopreserved sperm though not significantly so possibly given the sample size, and by day 14 the fertilisation rates were significantly lower than those obtained using cryopreserved spermatozoa (FIG. 7). The comparison between these two storage treatments using an IVF system is somewhat confounded by the fact that the act of freezing and thawing spermatozoa induces changes to the sperm membranes which mimic the process of capacitation; a final stage of maturation during which spermatozoa acquire the capacity to recognise, bind to, and fertilise the oocyte. For this reason, when spermatozoa are thawed they do not need to undergo the same extensive chemical-induced capacitation prior to co-incubation with the eggs that is required when fresh spermatozoa is utilised. Capacitation is a process which occurs naturally in the female reproductive tract following artificial insemination. The inventors though consider the lack of capacitation-like-changes in bovine spermatozoa stored in the New bovine sperm extender to be advantageous for sperm longevity post artificial insemination (AI).

In summary, in the present study bovine spermatozoa were stored at RT without the use of a specific device to control temperature. This markedly reduces the logistical constraints associated with the transport of chilled and cryopreserved spermatozoa for field use. Further, the New bovine extender medium allowed bull spermatozoa to be stored at room temperature for at least 10 days without any loss of functionality, and 7 days with equal or superior in vitro fertility compared to cryopreserved spermatozoa. It is further believed by the inventors that avoiding cryopreservation can improve the longevity of spermatozoa in the bovine reproductive tract following artificial insemination.

### Example 4: Artificial Insemination (AI) Study

The fertility of spermatozoa stored in New bovine sperm extender medium containing 82.2 mM glycine was confirmed in a small field trial. For this trial, bovine semen was collected via electroejaculation from 3 bulls; with high quality spermatozoa selected using a 45/90 discontinuous BoviPure™ density gradient, resuspended at a concentration of ˜50 million/mL in the New bovine extender medium, and stored in the dark at ambient temperature (˜22° C.). The stored spermatozoa showed no significant decline in total or progressive motility after 7 days (Total motility (TM): 83.3±3.06 vs. 73±3.18; Progressive motility (PM): 82.8±2.83 vs. 70.9±3.97; at Day 0 and Day 7 respectively, where P>0.05). At Day 7, fixed-time AI following oestrous synchronisation was performed on 18 2-year-old virgin heifers using an insemination dose of 0.5 mL (15.75-18.75×10⁶ progressively motile spermatozoa).

Ultrasonographic pregnancy diagnosis at 33 days post-insemination confirmed 14 of 18 females were in calf.

### Example 5: Further Artificial Insemination (AI) Study

A further fixed-time artificial insemination (AI) field trial was performed on 12 heifers following oestrous synchronisation. The trial was carried out near Cessnock, New South Wales, Australia. Briefly, semen was collected from a single Santa Gertrudis bull via electroejaculation and high-quality spermatozoa were selected and stored for 7 days at ambient room temperature (˜22° C.) at a concentration of ˜50 million/ml in New bovine extender medium containing 82.2 mM glycine as per the protocol described in Example 4. Again, the stored spermatozoa showed no significant decline in total or progressive motility after 7 days. Heifers were inseminated at Day 7 and at 35 days post-insemination, ultrasonographic pregnancy diagnosis showed 6 of the heifers were in calf. All of the pregnant heifers went on to calve at the end of August 2022. All calves were born live and healthy.

Santa Gertrudis is a taurine-indicine hybrid cattle breed descended from both zebu and European cattle and has strong Bos indicus traits. In the present trial, 3 of the pregnant heifers were pure-bred Santa Gertrudis, 2 were Bos taurus (Friesian crosses) and 1 was a Brahman/Santa Gertrudis cross.

This trial shows that Bos indicus sperm can be stored at ambient temperature for 7 days in the New bovine extender medium embodied by the present invention with retention of fertility, and that Bos indicus heifers can achieve pregnancy employing fixed-time AI utilising the sperm.

### REFERENCES CITED

Aitken J B, Naumovski N, Curry B, Grupen C G, Gibb Z, Aitken R J (2015) Characterization of an L-Amino Acid Oxidase in Equine Spermatozoa. Biology of Reproduction 92 (5) (125), 1-13.
Arias M E, Andara K, Briones E, Felmer R (2017) Bovine sperm separation by Swim-up and density gradients (Percoll and BoviPure): Effect on sperm quality, function and gene expression. Reproductive Biology.
Aurich C, Spergser J (2007) Influence of bacteria and gentamicin on cooled-stored stallion spermatozoa. Theriogenology 67 (5), 912-918.
Bergeron A, Manjunath P (2006) New insights towards understanding the mechanisms of sperm protection by egg yolk and milk. Molecular Reproduction and Development 73 (10), 1338-1344.
Biggers J D, Whitten W K, Whittingham D G (1971) The culture of mouse embryos in vitro. In ‘Methods in Mammalian Embryology.’ Ed. J C Daniels) pp. 86-116. (Freeman: San Fransisco)
Foote, R H (2002) Spermicidal effects of amphotericin B and nystatin on bull and rabbit sperm and contraceptive effects in rabbits. Contraception 66, 193-197.
Galuppo A G, Junior N B, Arruda N S, Corbellini A O, Chiappetta C M, Pavao D L, D'Angelo M, Canal C W, Rodrigues J L (2013) Evaluation of the effectiveness of semen processing techniques to remove bovine viral diarrhea virus from experimentally contaminated semen samples. J Virol Methods 187 (2), 443-8.
Gibb, Z, Lambourne, S R, Quadrelli, J, Smith, N D, Aitken, R J (2015) L-Carnitine and pyruvate are prosurvival factors during the storage of stallion spermatozoa at room temperature. Biology of Reproduction 93 (4), 1-9.
Gibb, Z, Lambourne, S R, Quadrelli, J, Aitken, R J (2016) Alternative osmolytes for the ambient temperature storage of stallion spermatozoa, In: 18th International Congress on Animal Reproduction. Tours, France.
Klein, E K, Gunn, A J, Stephen, C P, Swegen, A, Aitken, R J, Gibb, Z (2019) L-carnitine is a pro-survival factor for ambient temperature storage of bull spermatozoa, In: 50th Annual Scientific Meeting of the Society for Reproductive Biology (ESA-SRB-AOTA).’ Sydney, Australia, 18-21 Aug. 2019.
Le Guillou J, Ropers M H, et al. (2016) Sequestration of bovine seminal plasma proteins by different assemblies of phosphatidylcholine: A new technical approach. Colloids Surf B Biointerfaces 140, 523-530.
Lusignan M F, Bergeron A, Lafleur M, Manjunath P (2011) The major proteins of bovine seminal plasma interact with caseins and whey proteins of milk extender. Biol Reprod 85 (3), 457-64.
Murphy E M, Murphy C, O'Meara C, Dunne G, Eivers B, Lonergan P, Fair S (2017) A comparison of semen diluents on the in vitro and in vivo fertility of liquid bull semen. Journal of Dairy Science 100 (2), 1541-1554.
Murphy E M, Eivers B, O'Meara C M, Lonergan P, Fair S (2018) Effect of storage temperature, nitrogen gassing and sperm concentration on the in vitro semen quality and in vivo fertility of liquid bull semen stored in INRA96. Theriogenology 108, 223-228.
Ortiz-Rodriguez J M, Balao da Silva C, Masot J, Redondo E, Gazquez A, Tapia J A, Gil C, Ortega-Ferrusola C, Peña F J. Rosiglitazone in the thawing medium improves mitochondrial function in stallion spermatozoa through regulating Akt phosphorylation and reduction of caspase 3. PLOS ONE 2019; 14: e0211994.
Othman, F M, Mohamed, M I, El-Badry, DuDA, Shaker, M H, Kotb, MhR, Turky, H A (2016) Efficacy and safety of different antimycotic formulas used in sheep frozen semen extenders. Alexandria Journal for Veterinary Sciences 48, 77-82.
Samardzija M, Karadjole M, Getz I, Makek Z, Cergolj M, Dobranic T (2006a) Effects of bovine spermatozoa preparation on embryonic development in vitro. Reprod Biol Endocrinol 4, 58.
Samardzija M, Karadjole M, et al. (2006b) A comparison of BoviPure® and Percoll® on bull sperm separation protocols for IVF. Animal Reproduction Science 91 (3-4), 237-247.
Silver, I A, Erecińska, M (1997) Energetic demands of the Na+/K+ ATPase in mammalian astrocytes. Glia 21, 35-45.
Swegen A, Lambourne S R, Aitken R J, Gibb Z. Rosiglitazone Improves Stallion Sperm Motility, ATP Content, and Mitochondrial Function. Biol Reprod 2016; 95 (5): 107:1-12.
Upreti G C, Jensen K, Munday R, Duganzich D M, Vishwanath R, Smith J F (1998) Studies on aromatic amino acid oxidase activity in ram spermatozoa: role of pyruvate as an antioxidant. Animal Reproduction Science 51 (4), 275-287.
Varela E, Rey J, et al. (2018) How does the microbial load affect the quality of equine cool-stored semen? Theriogenology 114, 212-220.

The invention claimed is:

1. A sperm extender medium for prolonging the fertility of bovine spermatozoa during liquid storage of the spermatozoa, the medium comprising:
a monosaccharide energy source for the spermatozoa;
one or more α or β amino acid osmolytes selected from mono-amino carboxylic and mono-amino sulfonic acids, each independently having an R side group selected from the group consisting of H and non-polar, neutral open aliphatic chains excluding branched C3 and longer aliphatic chains;
one or more antioxidants; and
physiologically acceptable inorganic salts for maintaining sperm viability;
wherein the medium is buffered for maintaining pH of the medium in a range of from about 7.0 to about 8.5, the medium having a concentration of sodium ions of less than about 95 mM, and an osmolarity in a range of from about 290 mOsm/L to about 400 mOsm/L.

2. The sperm extender medium of claim 1, wherein each said amino acid osmolyte is independently unsubstituted or is monosubstituted with a single R side group of other than H.

3. The sperm extender medium of claim 1, wherein the R side group of each amino acid osmolyte is independently either H, an aliphatic C1-C2 group, or an unbranched C3 chain.

5. The sperm extender medium of claim 1, wherein each amino acid osmolyte independently has a formula selected from the group consisting of H₂N—CH₂—COOH, H₂N—CH(R)—COOH, H₂N—C₂H₃(R)—S(O)₂OH and H₂N—CH₂—CH₂—S(O)₂OH, wherein R is the R side group and is other than H.

6. The sperm extender medium of claim 1, wherein the one or more amino acid osmolytes comprises at least the majority of α and/or β amino acid(s) in the extender medium.

10. The sperm extender medium of claim 1, wherein the one or more amino acid osmolyte(s) is selected from the group consisting of glycine, L-alanine, L-valine, and combinations of the foregoing.

11. The sperm extender medium of claim 1, further comprising one or more agents selected from one or more of pyruvate, lactate, and further organic osmolytes optionally comprising L-carnitine.

13. The sperm extender medium of claim 1, further comprising a physiologically acceptable antihyperglycemic agent for enhancing the uptake of the monosaccharide energy source by the spermatozoa.

14. The sperm extender medium of claim 13, wherein the antihyperglycemic agent is rosiglitazone.

15. The sperm extender medium of claim 1, wherein the monosaccharide energy source is selected from the group consisting of fructose, glucose and combinations thereof and/or wherein the one or more antioxidants comprises coenzyme Q10 and/or further comprising one or more of spermatozoa anti-agglutination agent(s), acidity regulating agent(s), buffering agent(s), and antimicrobial agent(s).

18. The sperm extender medium of claim 1, wherein the physiologically acceptable salts are for providing ions selected from K⁺, Na⁺, Ca2⁺, Mg2⁺, Cl⁻, SO₄²⁻ and PO₄²⁻ in solution.

19. The sperm extender medium of claim 1, wherein the sperm extender medium is capable of maintaining fertility of the spermatozoa for a period of greater than 3 days in the absence of cryopreservation of the spermatozoa.

20. The sperm extender medium of claim 1, wherein the sperm extender medium is capable of for maintaining fertility of the spermatozoa for a period of at least 5 days in the absence of cryopreservation of the spermatozoa.

21. The sperm extender medium of claim 1, being for liquid storage of the spermatozoa at ambient temperature.

22. The sperm extender medium of claim 21, wherein the ambient temperature is in a range of from about 17° C. to about 25° C.

23. The sperm extender medium of claim 1, wherein the medium prolongs the fertility of spermatozoa of other livestock animals selected from sheep (Ovis aries) and goats (Capra hircus) in liquid storage.

24. The sperm extender medium of claim 1, wherein the bovine animal is a Bos taurus animal.

25. A preparation of spermatozoa of a livestock animal, the spermatozoa being in a sperm extender medium as defined in claim 1.

26. A method for fertilising an ovum of a livestock animal, comprising contacting the ovum with a spermatozoa of the animal administered in a sperm extender medium as defined in claim 1, the ovum and spermatozoa being of the same animal type.

28. A method for prolonging the fertility of spermatozoa of a livestock animal during liquid storage of the spermatozoa, comprising maintaining the spermatozoa in a sperm extender medium as defined in claim 1 for a period of at least 3 days.
